(19) United States
(12) Patent Application Publication
(10) Pub. No.: US 2021/0169117 A1
(43) Pub. Date: Jun. 10, 2021

(54) INFANT FORMULA

(71) Applicant: Arla Foods AMBA, Viby J (DK)

(72) Inventors: Esben Skipper Sorensen, Sabro (DK); Hans Burling, Lund (SE); Karen Bøttcher, Kibæk (DK); Hans Bertelsen, Videbæk (DK); Kristian Albertsen, Videbæk (DK); Gitte Graverholt, Arhus N (DK); Anders Steen Jørgensen, Arhus C (DK); Lotte Schack, Risskov (DK)

(21) Appl. No.: 17/119,879

(22) Filed: Dec. 11, 2020

Related U.S. Application Data
(63) Continuation of application No. 13/950,263, filed on Jul. 24, 2013, now abandoned.
Application No. 13/950,263 is a continuation of application No. 12/509,331, filed on Jul. 24, 2009, now abandoned.
Application No. 12/509,331 is a continuation of application No. 10/942,870, filed on Sep. 17, 2004, now abandoned.
(60) Provisional application No. 60/504,952, filed on Sep. 23, 2003.

(30) Foreign Application Priority Data
Sep. 18, 2003 (DK) PA 2003 01353

(51) Int. Cl. A23L 33/00; A61K 38/19

(52) U.S. Cl. CPC A23L 33/40; A61K 38/19

(57) ABSTRACT

Infant formula containing osteopontin and use of osteopontin as supplement for infant formulas or baby food.

### CROSS-REFERENCE TO RELATED APPLICATIONS

This application is a Continuation of and claims the benefit and priority to U.S. patent application Ser. No. 13/950,263, filed on Jul. 24, 2013, which is a Continuation of U.S. patent application Ser. No. 12/509,331, filed on Jul. 24, 2009, which is a Continuation of U.S. patent application Ser. No. 10/942,870, filed on Sep. 17, 2004, which claims the benefit of priority to U.S. Provisional Application Ser. No. 60/504,952, filed Sep. 23, 2003, and to Danish Patent Application Number PA 2003 01353 filed Sep. 18, 2003. The disclosures of the above-referenced applications are hereby expressly incorporated by reference in their entireties.

### REFERENCE TO SEQUENCE LISTING

A Sequence Listing submitted as an ASCII text file via EFS-Web is hereby incorporated by reference in accordance with 37 U.S.C. § 1.52(e). The name of the ASCII text file for the Sequence Listing is SeqList-ZACCO118-002C2.txt, the date of creation of the ASCII text file is Dec. 11, 2020, and the size of the ASCII text file is 6 KB.

### FIELD OF THE INVENTION

The present invention relates to infant formulas or baby food supplemented with osteopontin and use of osteopontin in infant formulas or baby food.

### BACKGROUND OF THE INVENTION

It is well known that infant formulas do not possess the same immune stimulating effect as mother milk. Therefore breast fed babies have less infections together with less development of allergy and eczema compared to bottle fed babies. For this reason infant formulas are attempted to resemble human milk as much as possible. This is called to “humanize” infant formulas.

Osteopontin (OPN) is a protein present in milk from milk producing mammals, e.g. human and bovine milk.

So far it has been believed that human milk and bovine milk contained OPN in nearly the same concentration. Therefore it has not been proposed to add extra OPN to infant formulas in order to humanize infant formulas.

The content of OPN in human milk was estimated to be about 3-10 mg/litre. Surprisingly it has now been found that human milk on an average contains about 25-300 mg/litre, i.e. about 5-10 times as much OPN as bovine milk. Individual differences occur.

It has also been shown that OPN possesses a key function in the acquisition of Th-1-response. Infants possess from birth mainly Th-2 responses, due to the antibodies received from the mother and the maturation of the immune response in infants is mediated by the induction/acquisition of Th-1 responses. OPN is therefore believed to be an essential component in human milk for infant nutrition.

OPN is a multifunctional protein, involved in both physiological and pathological processes in multiple organs and tissues including biomineralization, inflammation, leukocyte recruitment and cell survival (reviewed in; Mazzali M, Kipari T, Ophascharoensuk V, Wesson J A, Johnson R, Hughes J.2002.Osteopontin—a molecule for all reasons. Q. J. 95:3-13; Denhart D T, Giachelli C M, Rittling SR.2001. Role of osteopontin in cellular signaling and toxicant injury. 41:4723-49). OPN is expressed by a number of cell types and epithelial cells. Accordingly, OPN is present in most tissues and organs that have been analyzed for its presence. OPN is present in relatively high concentrations in many body fluids, such as blood, plasma, bile, urine and milk. OPN exists in tissue-specific isoforms, e.g. proteolytically truncated forms, phosphorylation- and glycosylation variants etc., which may correspond to particular cellular functions.

Bovine milk OPN, the most well characterized form of the protein, contains 27 phosphoserines, one phosphothreonine and three 0-glycosylated threonines (Sørensen E S, Højrup P, Petersen T. 1995. Posttranslational modifications of bovine osteopontin: Identification of twenty-eight phosphorylation and three 0-glycosylations sites. Protein Sci. 4:2040-2049). All phosphorylations are situated in acidic recognition sequences of the mammary gland casein kinase and casein kinase 2 (Sørensen E S, Petersen T E. 1994. Identification of two phosphorylation motifs in bovine osteopontin. Biochem. Biophys. Res. Comm. 198:200-205).

Bovine and human OPN are very homologous with all functional elements being retained among the species. An alignment of the two proteins is shown in FIG. 1. The Arg-Gly-Asp integrin binding sequence is responsible for binding to cell receptors, the poly aspartic acid rich region is involved in the binding to hydroxyapatite, calcium salt and ions, and most interestingly almost all serine residues located in the recognition sequence of the mammary gland casein kinase shown to phosphorylate the bovine OPN is also present in the human sequence. Likewise, the threonine residues shown to be glycosylated in the bovine protein is also present in the human counterpart. This preservation of sites and motifs of posttranslational modification indicates that bioactive peptides that can be formed by digestion of the human OPN are most likely also formed by digestion of the bovine OPN. Therefore, the bovine protein is believed to be highly suitable to fortify e.g. infant formulas and baby food.

WO 02/28413 discloses the purification of an acidic protein fraction of milk protein by anion-exchange and the use of this fraction in bone health compositions. The described fraction contains a number of minor acidic whey proteins. These include proteose peptione component 3, proteose peptone component 5 (PP5), also known as beta-casein-5P (fl-105) or beta-casein-5P (fl-107), proteose peptone 8-slow (PP8-slow), also known as beta-casein-1P (f29-105) or beta-casein-1P (f29-107), sialyated and phosphorylated proteins alpha-s1-casein phosphopeptides and osteopontin, and also a mixture of peptides derived from these proteins as well as small amounts of beta-lactoglobulin, bovine serum albumin and immunoglobulins. The protein fraction described in the invention may be used for the generation of functional foods for the treatment and/or prevention of bone defects. The patent specification does not identify osteopontin as being the active component in the protein fraction, and the patent specification does not mention the use of osteopontin or the protein fraction described for immune stimulating purposes or for humanization of infant formulas.

Senger D R, Perruzzi C A, Papadopoulos A, Tenen D G. 1989. Biochem. Biophys. Acta. 996: 43-48. Purification of a human milk protein closely similar to tumor secreted phosphoproteins and osteopontin, describes the presence and purification of osteopontin in human breast milk. The authors estimate the concentration of osteopontin in human milk to be in the range of 3-10 mg/litre. This estimate is more than 10-fold below the value we measure in the present invention.

Dhanireddy R, Senger R, Mukherjee B B, Mukherjee A B. 1993. Acta Paediatr. 82:821-2. Osteopontin in human milk from mothers of premature infants, describes the presence of osteopontin in preterm milk. The milk samples are characterized by Western blotting. The concentrations of OPN in the samples are not determined. The presence of OPN in milk is only discussed in relation to calcium transport.

Sørensen S, Justesen S J, Johnsen, A H. 2003. Protein Expression and Purification 30:238-245. Purification and characterization of osteopontin from human milk, describes a new protocol for purification of human milk osteopontin. The article describes the purification of several fragments and peptides derived from osteopontin as well as intact full-length osteopontin. The estimation of the concentration of osteopontin in human milk is based on the amount purified by the procedure, and is roughly in agreement with the findings of Senger et al. 1989. This is more than 10-fold below the osteopontin value we measure by sandwich ELISA in the present invention. The article also describes the production of polyclonal antibodies against osteopontin, but these are not used to determine the actual amount of osteopontin in milk. The article simply describes a way of preparing osteopontin and osteopontin variants for standardization procedures, antibody production and functional studies. The article does not discuss or mention the function of, or potential use of osteopontin in relation to milk, infant formulas or any other food product.

Assays determining the OPN concentration in prior art estimates the content in the range of 3-10 mg/litre. However assays determining the OPN concentration in milk are difficult to perform due to, e.g. the interference of proteins forming aggregates. Therefore the OPN content in milk has not been determined precisely. Due to this OPN has not been added to infant formulas, in order to obtain a product which resembles mother milk with the beneficial protective effect against the development of allergy. Therefore infant formulas are currently not provided with immune stimulatory factors, which can increase the acquisition of Th1 responses.

### SUMMARY OF THE INVENTION

The invention is based on the surprising discovery that OPN is present in elevated levels in human breast milk compared to bovine milk. Previous estimations of the OPN content in milk have been based on Western blotting analyses and protein purification, i.e. 3-10 mg/litre. The development of a quantitative ELISA directed against milk OPN has lead to new insight into the concentration of OPN in milk.

Accordingly the present invention provides infant formulas supplemented with milk osteopontin of mammalian origin, human and non-human. In all known infant formulas OPN can be included as supplement or OPN can be given to the infant separately from the formula which promote acquisition of Th1 response and thus reduce impaired immune function in infants.

The amount of osteopontin given to the infant, either in an infant formula or separately from an infant formula can be comparable to that found in human breast milk. The osteopontin may be incorporated into all human infant formulas, such as bovine milk based and soy based infant formulas and in baby food such as NAN® from Nestle.

Infant formulas manufactured as starter formulas, follow-on formulas and LBW formulas can all comprise milk OPN.

Infant formulas should preferably comprise milk OPN in an amount of at least 1% of the total protein content corresponding to the amount of milk OPN in human breast milk.

### BRIEF DESCRIPTION OF FIGURES

FIG. 1 shows an alignment of bovine and human milk OPN. Phosphorylated residues in bovine OPN are shown in bold letters.

FIG. 2 shows IL-12 expression in human intestinal T cell after stimulation with bovine OPN.

### DETAILED DESCRIPTION OF PREFERRED EMBODIMENTS

In a large number of newborn infants who are not nursed by the mother but fed by an infant formula the acquisition of Th1 immune response may be delayed, thereby increasing the risk of allergy reactions. By giving the non-nursed new-born infant formulas supplemented with milk OPN, in amounts comparable to human milk, the induction of Th1 immune response is likely to occur.

Thus the invention proposes an infant formula or baby food supplemented with osteopontin.

Infant formulas preferably comprise osteopontin obtained from human or bovine milk. However, infant formulas can also be supplemented with osteopontin from milks of other animals such as goat, sheep, camel, dromedary or llama.

Preferably infant formulas comprise osteopontin in an amount corresponding to 1% of the total protein content. As much as 3% can be used, and preferably used amounts are more than 1.5%, 2% or 2.5%. Such an amount results in at least 25-300 mg/litre of milk osteopontin in the ready to feed formula. 50-250 mg/litre are preferred, especially 100-200 mg/litre. 130-150 mg/litre is most preferred. In many cases 130 mg/litre is believed to give the best infant formulas.

Another aspect of the present invention is the use of milk osteopontin as supplement in infant formulas or baby food.

Also certain immune deficiencies are believed to be treated by giving formulas with an extra supplement of milk osteopontin.

Milk osteopontin can as mentioned be obtained from all mammalian, such as camel, goat, sheep, llama and dromedary. Osteopontin is preferably obtained by purification from human or bovine milk.

Osteopontin can be applied to all known infant formulas, i.e. starter formulas, follow on formulas and LBW-formulas and premature formulas.

The concentration of milk osteopontin can depend on geographical position, ethnical group, lifestyle, food etc.

Milk osteopontin can also be applied to infant formulas given to other mammals than humans, such as domesticated animals, livestocks (pigs, cows, horses, etc.) pets and zoo animals.

Milk taken at different day's post-partum can be used as a measurement for the osteopontin concentration in human breast milk at different times post partum. This measurement can be used to have different OPN levels in, e.g. starter formulas; follow on formulas and LBW-formulas.

It is also possible to measure the OPN content in a mother's milk and supplement it with extra milk OPN if necessary and give it to her own child or another child.

The measurement of OPN in human milk may be carried out using ELISA or other immunological procedures such as Western blotting analyses.

An ELISA can be carried out in a number of different embodiments, many of which are applicable in the present invention. One ELISA embodiment, which is particularly suitable for use in the present invention, use antibodies specific for native milk OPN.

The infant formulas of the present invention are supplemented with milk OPN isolated from milk, however it will be appreciated that milk OPN may be prepared by recombinant means in appropriate cells yielding a phosphorylation pattern as found in the ‘milk-OPN’. Preferred cells for expression are those of the mammary gland, since these cells may be expected to yield an identical or essentially identical phosphorylation pattern.

It is well know that infant formulas do not possess the same immune stimulating effect as mother milk and breast fed babies are having less infections together with development of allergy and eczema compared to bottle fed babies. One reason could be that breast milk is containing high amounts of OPN.

### Definitions

As used herein the term “osteopontin” or “OPN” is used for osteopontin from milk, including naturally occurring fragments or peptides derived from OPN by proteolytic cleavage in the milk, or splice-, phosphorylation-, or glycosylation variants as obtainable from the method proposed in WO 01/49741.

Milk OPN is OPN purified and obtained from mammalian milk samples.

Mammalian milk is milk from humans and any milk producing animals, such as cows, camels, goats, sheep, dromedaries and llamas.

Breast milk: Milk collected from (healthy) human mothers.

Infant formulas are formulas which cover the nutrition need of an infant.

Starter formulas: formulas for babies which cover their nutrition needs within the first 4-6 months.

Follow-on formulas: formulas for babies which cover their nutrition needs after the first 4 months.

LBW (low birth weight) formulas: Formulas which cover the nutrition needs of an infant with low birth weight.

Premature formulas: Formulas which cover the nutrition needs of a premature infant.

The invention is further illustrated referring to the following non-limiting examples.

### EXAMPLES

### Collection and Treatment of Milk Samples

Human breast milk samples were collected from 10 healthy mothers 10-58 days post partum. The milk was collected by breast pump and 2 ml were sampled from each milking and pooled to obtain whole day milk. The cream was extracted from whole milk by high speed centrifugation and separated from skimmed milk. The skimmed milk was aliquoted into 0.2 ml fractions and analyzed immediately or frozen at −20° C. until required.

Fresh pooled cow's milk was obtained at a local dairy. Milk was obtained four times over a period of 4 months and processed individually. The cream was removed by centrifugation, and samples of the milk were frozen at −20° C. until required.

### Measurement of Total Protein Concentration in Milk

The protein concentration in all milk samples was determined by Bradford analysis using bovine serum albumin as standard. Analyses were performed according to the descriptions supplied by the manufacturer.

### ELISA for OPN in Milk

The concentration of OPN in the individual milks and in commercially available infant formulas was measured by a sandwich ELISA. For this assay, antiserum against bovine and human OPN against native OPN purified from bovine and human milk, respectively, were raised in rabbits at Dako (Glostrup, Denmark). The bovine OPN was purified essentially as described (Sørensen E S, Petersen T E, 1993, Purification and characterization of three proteins isolated from the proteose peptone fraction of bovine milk, J. Dairy Res. 60:189-197). Human milk OPN was purified essentially as described (Senger D R, Perruzzi C A, Papadopoulos A, Tenen D G, 1989, Purification of a milk protein closely similar to tumor-secreted phosphoproteins and osteopontin. Biochem. Biophys. Acta 996:43-48). For extra high purity both proteins were subjected to reverse-phase chromatography before immunization of the rabbits. The IgG fraction of the antiserum was purified by Concavalin A affinity and used directly in the ELISA, or purified on an OPN affinity column to obtain highly specific antibodies. The specificities of the antibodies were checked and verified by Western blotting analyses of milk samples and purified OPN. The developed ELISA was very sensitive with a detection limit <5 ng/ml milk, and linearity in the range of 10-300 ng/ml, accordingly milk samples were diluted 2000-23000 times before analyses. All samples were analyzed in triplicates in 6-12 dilutions per measurement. The reliability of the ELISA was checked by spiking milk samples with the known amounts of OPN showing that the assay was quantitative with full recovery of the spiked amount of OPN in the range used for these measurements. Likewise, purification of OPN from human milk samples showed OPN yield comparable with the concentrations measured by the ELISA.

### Concentration of OPN in Bovine Milk

The level of OPN in pooled bovine milk was determined four times during a 4 months period (Table 1). The average OPN concentration was found to be 18.28 mg/litre. The total protein was set to 35,000 mg/litre according to the literature. The OPN content in bovine milk is 0.05% (w/w) of the total protein.

TABLE 1
OPN concentration in bovine milk

| Bovine milk | OPN | Total protein | OPN/Protein |
|---|---|---|---|
| ‘sample no.’ | (mg/litre) | (mg/litre) | (%) |
| 301002 | 17.84 | | |
| 061102 | 19.37 | | |
| 141101 | 17.91 | | |
| 120203 | 18.01 | | |
| Average | 18.28 | ~35,000 | 0.052 |

### Concentration of OPN in Human Milk

The milk from 10 mothers, aged 25-35 years (average 29 years) was sampled 10-58 days post partum (average 21.6 days post partum) and analyzed for OPN and total protein. The data are summarized in table 2. The OPN in the breast milk ranged from 22.44 mg/litre to 257.35 mg/litre with an average value of 138.5 mg/litre. The total protein concentration in the milk ranged from 8327 mg/litre to 13268 mg/litre with an average value of 10821 mg/litre. The OPN contributes an average of 1.3% (w/w) of the total protein in human milk and in a single mother more than 3% of the milk protein was OPN.

TABLE 2
OPN concentration in human milk.

| | | | OPN | Total protein | OPN |
|---|---|---|---|---|---|
| | Age of | Days post | Concentration | concentration | protein |
| | mother | partum | (mg/litre) | (mg/litre) | (%) |
| | 28 | 59 | 65.97 | 8550 | 0.77 |
| | 35 | 13 | 257.35 | 8327 | 3.09 |
| | 28 | 12 | 200.52 | 13268 | 1.50 |
| | 30 | 10 | 238.69 | 12628 | 1.89 |
| | 25 | 12 | 22.44 | 11442 | 0.20 |
| | 30 | 33 | 101.54 | 10000 | 1.02 |
| | 31 | 39 | 113.96 | 10157 | 1.12 |
| | 30 | 17 | 67.04 | 10328 | 0.65 |
| | 23 | 12 | 212.00 | 10936 | 1.94 |
| | 30 | 10 | 105.00 | 12576 | 0.83 |
| Average | 29 | 21.6 | 138.4 | 10821 | 1.3 |

There is a considerable variation in the OPN concentration related to total protein in individual mothers. In all mothers the OPN/total protein ratio was significantly higher than the corresponding value for bovine milk. These measurements show that the average OPN concentration related to total protein in human milk (1.3%) compared to bovine milk (0.05%) is about 26 times higher. This average value covers individual variations in the range 4 to 62 times the bovine value.

### Concentration of OPN in Commercially Available Infant Formulas

Data are calculated as mg OPN/liter ready to feed formula, based on 125 g/liter.

TABLE 3

| From the firm | Infant Formula | Content of OPN, mg/liter |
|---|---|---|
| Nam Yang | Science 1 | 11.5 |
| | Science 2 | 9.4 |
| | Agisarang 3 | 12.3 |
| | Agisarang 4 | 17.4 |
| | XO1 | 7.9 |
| | XO2 | 6.2 |
| | XO3 | 8.4 |
| | XO4 | 6.7 |
| | Agisarang soo | 5.5 |
| | Imperial Dream | 6.0 |
| | Science | 17.8 |
| | Premium XO | 13.0 |
| Nestlé | Nan 1 | 10.3 |
| | Nidina 1 | 5.3 |
| Beauvais | Allomin 2 | 6.4 |

### Bovine Osteopontin Induces IL-12 Expression in Human Intestinal Gut Cells

Human intestinal cells were obtained and cultured in the presence of IL-2 and IL-4 essentially as described (Agnholt J, Kaltoft K, 2001. Infliximab downregulates interferon production in activated gut T-lymfocytes from patients with Crohn's disease. Cytokine. 15:212-222). The cultivation in the presence of IL-2 and IL-4 promote the growth of T-cells with preserved cellular features such as expression of the receptors; TCRαβ+, CD4+CD45RO+.

Cell culture wells were coated with 1 ml (1 mg/ml) osteopontin PBS-solution and PBS, respectively, over night at 4° C. The wells were emptied and T-cells were cultured in the wells (10⁶ cells/ml) for 24 hours and subsequently aliquots of the cell supernatants were sampled for analyses.

Cytokine-matched antibody pairs for determination of IL-12 were obtained from R&D Systems. The detecting antibodies were all biotinylated. A time-resolved fluorometric assay applying Europium (Eu³⁺) labelled streptavidin and a Delphia 1234 fluorometer (Wallac, Turku, Finland) were used to determine the IL-12 content. Obtained values are averages of three ELISA readings in triplicate experiments.

TABLE 4
IL-12 expression in human gut immune after
stimulation with bovine osteopontin

| | IL-12 (pg/ml) |
|---|---|
| | Average (std. dev.) |
| Control cells | 3.3 (+/−3.2) |
| +Osteopontin | 16.7 (+/−2.1) |

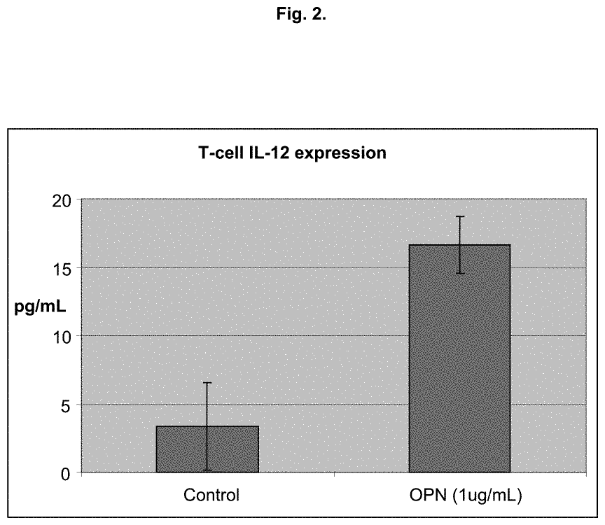

As shown in table 3 and FIG. 2, a significant raise in the expression of IL-12 can be observed in the human immune cells stimulated with native bovine osteopontin.

Here we show that native bovine OPN is able to induce expression of the Th-1 cytokine IL-12 in non-activated human intestinal immune cells, strongly indicating a role in the regulation of intestinal immunity.

### Mix of Infant Formulas Comprising OPN

A level of 100-130 mg/litre OPN is considered a suitable amount for infant formulas in the following examples. However, the scope of the present invention is not limited to this amount since variation in human milk compositions due to food, lifestyle, geographical area, ethnical group etc exist.

Incorporated in the examples of the present invention are infant formula compositions, which have been prepared, based on EU legislation and recommendations for infant formulas.

In connection with this we only take the protein amount in account, since the other parts of the product are expected to be unchanged. We have chosen to apply 100 mg OPN/1000 ml to all products, however it is also possible and within the scope of the present invention to apply OPN according to the protein level per se.

Cow milk contains OPN in the range of 15-20 mg/litre which is at least 5 times below the OPN content in human milk measured in the examples in this invention to an average of 138.4 mg/litre. OPN in the range of 15-20 mg/litre corresponds to 0.05% of the total protein content in bovine milk. This figure is approximately 26 times lower than 1.3% (OPN/total protein) as measured in human milk in the examples in this invention.

We have in our calculations assumed that OPN is contained in the whey part of the milk and the level will therefore be approximately 18 mg/100 g milk powder and approximately 360 mg/100 g WPC80 powder. Due to these numbers we have calculated the level, which is expected to arise from the used ingredients.

In some of the following examples we have reduced the part of whey protein by the additional OPN amount, since the glycoprotein is considered to follow whey under the precipitation of rennet-casein at approximately pH 6.2 and acid casein at pH 4.6.

The protein level for infant formulas can be calculated as follows:

### Starter Formulas

Starter formulas mean food for babies which cover their nutrition needs within the first 4-6 months.

Regulatory Requirements for the Protein Content within EU.

Source: Announcement no. 202 regarding infant formulas and supplements for newborn infants and babies:

### Proteins:

Protein content=nitrogen content×6.38 when cow milk proteins

Protein content=nitrogen content×6.25 when soy milk proteins isolator and partly hydrolyzed protein.

By chemical index is meant the lowest ratios between the amount of essential amino acid in the present protein and the amount of the corresponding amino acid in the reference protein.

Products based on cow milk:

| | Minimum | Maximum |
|---|---|---|
| Starter formulas | 0.45 g/100 kJ | 0.7 g/100 kJ |
| | (1.8 g/100 kcal) | (3 g/100 kcal) |

At the same energy content the product should contain an accessible amount of essential and semi essential amino acids as the reference protein (mother milk as defined in annex 6); at the calculation the methionin and cystein content can be added.

Products based on partly hydrolyzed protein:

| | Minimum | Maximum |
|---|---|---|
| Starter formulas | 0.56 g/100 kJ | 0.7 g/100 kJ |
| | (2.25 g/100 kcal) | (3 g/100 kcal) |

At the same energy content the product should contain an accessible amount of essential and semi essential amino acids as the reference protein (mother milk as defined in annex 6); at the calculation the methionin and cystein content can be added.

Protein effectiveness ratio (PER) and netto protein utilization (NPU) should correlate at least to casein. Taurin content has to be at least 10 μmol/100 kJ (42 μmol/100 kcal) and the L-carnithin content should at least be 1.8 μmol 100 kJ (7.5 μmol/100 kcal).

Products based solely on soy protein isolates or in compositions together with cow milk proteins:

| | Minimum | Maximum |
|---|---|---|
| Starter formulas | 0.56 g/100 kJ | 0.7 g/100 kJ |
| | (2.25 g/100 kcal) | (3 g/100 kcal) |

Only soy protein isolates can be used for these infant formulas. The chemical index of the proteins should be at least 80 percent of the reference protein (mother milk defined in annex 7 in the announcement).

At the same energy content the product should contain at least an accessible amount of methionin comparable to the reference protein (mother milk). The content of L-carnithin should be at least 1.8 μmol/100 kJ (7.5 μmol/100 kcal).

Amino acids can only be applied to products in order to enhance the nutritional value and only in ratios, necessary to reach the aim.

Examples: mix for starter formulas

Energy: 2200 kJ/100 g/525 kcal/100 g)

Reconstitution: 125 g per litre

Traditional infant starter formula

| | Powder | Reconstituted | | |
|---|---|---|---|---|
| | gram per | gram per | Gram per | Gram per |
| | 100 g | litre | 100 kcal | 100 kJ |
| Total protein* | 12 | 15 | 2.29 | 0.550 |
| Casein | 4.8 | 6.1 | 0.91 | 0.220 |
| Whey protein | 7.2 | 9.1 | 1.38 | 0.330 |
| *of this OPN | 0.030 | 0.038 | 0.006 | 0.001 |
| Ratio Casein/ | 40/60 | | | |
| Whey protein | | | | |

Infant starter formula comprising OPN

| | Powder | Reconstituted | | |
|---|---|---|---|---|
| | gram per | gram per | Gram per | Gram per |
| | 100 g | litre | 100 kcal | 100 kJ |
| Total protein | 12 | 15 | 2.29 | 0.550 |
| Casein | 4.80 | 6.1 | 0.91 | 0.220 |
| *of this OPN | 0.030 | 0.038 | 0.006 | 0.001 |
| Whey protein | 7.12 | 9.0 | 1.36 | 0.326 |
| Added OPN | 0.074 | 0.092 | 0.014 | 0.003 |
| Ratio casein/ | 40/60 | | | |
| whey protein | | | | |

### Follow-on Formulas

Regulatory Requirements for the Protein Content within EU.

Source: Announcement no. 202 regarding infant formulas and supplements for newborn infants and babies:

### Proteins:

Protein content=nitrogen content×6.38 when cow milk proteins

Protein content=nitrogen content×6.25 when soy milk proteins isolator

By chemical index is meant the lowest ratios between the amount of essential amino acid in the present protein and the amount of the corresponding amino acid in the reference protein.

Products based on cow milk:

| | Minimum | Maximum |
|---|---|---|
| Starter formulas | 0.50 g/100 kJ | 1 g/100 kJ |
| | (2.25 g/100 kcal) | (4.5 g/100 kcal) |

The chemical index of the proteins should be at least 80 percent of the reference protein (mother milk defined in annex 7 in the announcement). Only soy protein isolates can be used for these infant formulas. Amino acids can only be applied to products in order to enhance the nutritional value and only in ratios necessary to reach the aim.

At the same energy content the product should contain an accessible amount of essential and semi essential amino acids as the reference protein (mother milk as defined in annex 6); at the calculation the methionin and cystein content can be added.

### Examples

Energy: 2170 kJ/100 g (520 kcal/100 g)

Reconstitution: 150 g per litre

Traditional follow-on formulas

| | Powder | Reconstituted | | |
|---|---|---|---|---|
| | gram per | gram per | Gram per | Gram per |
| | 100 g | litre | 100 kcal | 100 kJ |
| Total protein * | 15 | 22.5 | 2.89 | 0.690 |
| Casein | 12 | 18 | 2.31 | 0.554 |
| Whey protein | 3 | 4.5 | 0.58 | 0.138 |
| *of this OPN | 0.007 | 0.011 | 0.001 | 0.000 |
| Ratio casein/ | 80/20 | | | |
| whey protein | | | | |

Follow-on formula comprising OPN

| | Powder | Reconstituted | | |
|---|---|---|---|---|
| | gram per | gram per | Gram per | Gram per |
| | 100 g | litre | 100 kcal | 100 kJ |
| Total protein | 15 | 22.5 | 2.89 | 0.690 |
| Casein | 12 | 18 | 2.31 | 0.554 |
| Whey protein | 2.92 | 4.4 | 0.56 | 0.134 |
| *of this OPN | 0.007 | 0.011 | 0.001 | 0.000 |
| OPN | 0.078 | 0.117 | 0.015 | 0.004 |
| Ratio Casein/ | 80/20 | | | |
| Whey protein | | | | |

### Infant Formulas for Low-Birth Weight (LBW) Babies

Presently there is no legislation in this area, however it is under process. Manufacturers of infant formulas for LBW babies are currently getting recommendations from pediatrists and “IDACE proposal for guidelines on compositions of low-birth-weight formulae for marketing in the European Union”. These guidelines are expected to provide the base for the upcoming legislation in EU.

Proteins:

Protein content=nitrogen×6.38 for cow milk and hydrolyzed cow milk proteins.

Products based on cow milk and hydrolyzed cow milk proteins.

| | Minimum | Maximum |
|---|---|---|
| Low-birth-weight | 0.6 g/100 kJ | 0.8 g/100 kJ |
| | (2.4 g/100 kcal | (3.3 g/100 kcal) |

The formulas have to contain an accessible amount of each essential and semi essential amino acid at least comparable to the content in the reference protein (breast milk). In contrast to standard infant formulas the amount of methionin and cystein cannot be added for calculation purposes

The taurine content should be at least 1.3 mg/100 kJ (5.3 mg/100 kcal).

### Example

Energy: 315 kJ/100 ml (75 kcal/100 ml) Reconstitution: 150 g per litre

### Traditional LBW Formula

| | Powder | Reconstituted | | |
|---|---|---|---|---|
| | gram per | gram per | Gram pr. | Gram pr. |
| | 100 g | litre | 100 kcal | 100 kJ |
| Total protein * | 15 | 22.5 | 3 | 0.718 |
| Hydrolyzed | 14.96 | 22.44 | 2.992 | 0.716 |
| whey protein | | | | |
| *of this OPN | 0.067 | 0.101 | 0.012 | 0.002 |
| Taurine | 0.04 | 0.06 | 0.008 | 0002 |

### LBW Formula Comprising OPN:

| | Powder | Reconstituted | | |
|---|---|---|---|---|
| | gram per | gram per | Gram per | Gram per |
| | 100 g | litre | 100 kcal | 100 kJ |
| Total protein * | 15 | 22.5 | 3.000 | 0.718 |
| Hydrolyzed | 14.89 | 22.34 | 2.972 | 0.712 |
| whey protein | | | | |
| *of this OPN | 0.067 | 0.101 | 0.012 | 0.002 |
| Taurine | 0.04 | 0.06 | 0.008 | 0.002 |
| OPN added | 0.023 | 0.034 | 0.005 | 0.002 |

### FIGURES

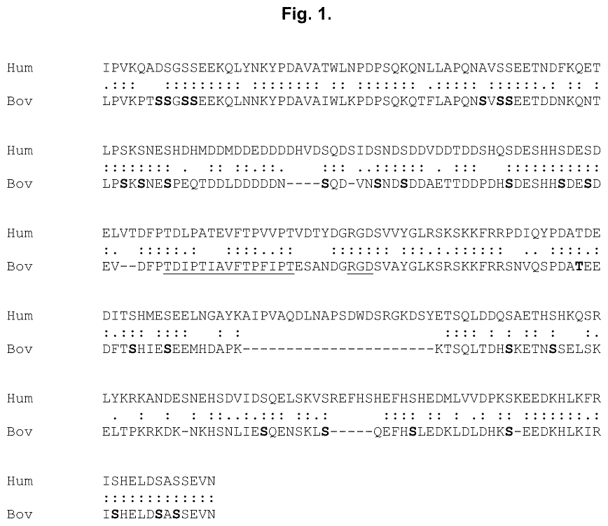

FIG. 1.

Alignment of human and bovine OPN. Identity is indicated by two dots and homologous amino acids are indicated by a single dot. Phosphorylated residues in bovine OPN are shown in bold. The RGD (Arg-Gly-Asp) integrin binding triplet and the region containing the glycosylations in the bovine sequence are underlined

FIG. 2.

IL-12 expression of T-cells stimulated with bovine OPN.

1. (canceled)

2. An improved pasteurized starter or follow-on infant formula, wherein the improved pasteurized starter or follow-on infant formula is a bovine milk based infant formula, wherein the improved pasteurized starter or follow-on infant formula further comprises a non-human mammalian milk osteopontin in a final concentration of 50-250 mg/litre, wherein the non-human mammalian milk osteopontin is purified and obtained from bovine milk, and wherein said improved pasteurized starter or follow-on infant formula is a ready to feed human infant formula or can be mixed with water to a reconstituted human infant formula.

3. The improved pasteurized starter or follow-on infant formula of claim 2, wherein the addition of said non-human mammalian milk osteopontin is in the form of a powder.

4. The improved pasteurized starter or follow-on infant formula of claim 2, wherein the addition of said non-human mammalian milk osteopontin is in the form of a liquid.

5. The improved pasteurized starter or follow-on infant formula of claim 2, wherein said final concentration is 100-200 mg/litre of nonhuman mammalian milk osteopontin.

6. The improved pasteurized starter or follow-on infant formula of claim 2, wherein said final concentration is 130-150 mg/litre of nonhuman mammalian milk osteopontin.

7. The improved pasteurized starter or follow-on infant formula of claim 2, wherein said final concentration is 100-130 mg/litre of nonhuman mammalian milk osteopontin.

8. The improved pasteurized starter or follow-on infant formula of claim 2, wherein said final concentration is 65-130 mg/litre of nonhuman mammalian milk osteopontin.

9. The improved pasteurized starter or follow-on infant formula of claim 2, wherein said final concentration is 130 mg/litre of non-human mammalian milk osteopontin.

10. The improved pasteurized starter or follow-on infant formula of claim 2, wherein the formula comprises 1-3% osteopontin by weight of total protein of the improved starter or follow-on infant formula.

11. The improved pasteurized starter or follow-on infant formula of claim 2, wherein the infant formula is a starter formula.

12. The improved pasteurized starter or follow-on infant formula of claim 2, wherein the infant formula is a follow-on formula.

13. A humanized and pasteurized starter or follow-on infant formula composition comprising:
a) a pasteurized starter or follow-on infant formula, wherein the pasteurized starter or follow-on infant formula is a bovine milk based infant formula; and
b) 50-250 mg of non-human mammalian milk osteopontin per liter of said humanized and pasteurized starter or follow-on infant formula, wherein the non-human mammalian milk osteopontin is purified and obtained from bovine milk,
wherein said humanized and pasteurized starter or follow-on infant formula is a reconstituted or a ready to feed human infant formula.

14. The humanized and pasteurized starter or follow-on infant formula composition of claim 13, comprising said non-human mammalian milk osteopontin dissolved from powder or liquid.

15. The humanized and pasteurized starter or follow-on infant formula composition of claim 13, comprising 100-200 mg of the nonhuman mammalian milk osteopontin per liter of said reconstituted or ready to feed infant formula.

16. The humanized and pasteurized starter or follow-on infant formula composition of claim 13, comprising 130-200 mg of the nonhuman mammalian milk osteopontin per liter of said reconstituted or ready to feed infant formula.

17. The humanized and pasteurized starter or follow-on infant formula composition of claim 13, comprising 100-130 mg of the nonhuman mammalian milk osteopontin per liter of said reconstituted or ready to feed infant formula.

18. The humanized and pasteurized starter or follow-on infant formula composition of claim 13, comprising 65-130 mg of the nonhuman mammalian milk osteopontin per liter of said reconstituted or ready to feed infant formula.

19. The humanized and pasteurized starter or follow-on infant formula composition of claim 13, comprising 130 mg of the nonhuman mammalian milk osteopontin per liter of said reconstituted or ready to feed infant formula.

20. The humanized and pasteurized starter or follow-on infant formula composition of claim 13, comprising 138 mg of the nonhuman mammalian milk osteopontin per liter reconstituted or ready to feed infant formula.

21. The humanized and pasteurized starter or follow-on infant formula composition of claim 13, wherein the infant formula is in the form of a powder for reconstitution before feeding.

22. The humanized and pasteurized starter or follow-on infant formula composition of claim 13, wherein the infant formula is a starter formula.

23. The humanized and pasteurized starter or follow-on infant formula composition of claim 13, wherein the infant formula is a follow-on formula.

24. The humanized and pasteurized starter or follow-on infant formula composition of claim 13, wherein the composition comprises 1-3% osteopontin by weight of total protein of the infant formula composition.

25. A process for producing a humanized and pasteurized infant formula, the process comprising:
a) providing a pasteurized infant formula in dry powder form, wherein the pasteurized infant formula is a bovine milk based infant formula; and
b) adding non-human mammalian milk osteopontin to the provided pasteurized infant formula, wherein the non-human mammalian milk osteopontin is purified and obtained from bovine milk, the osteopontin provided in dry powder form;
wherein the amount of added osteopontin provides 50-250 mg/liter of the osteopontin when reconstituted in a ready to feed infant formula.

26. A process for producing an improved pasteurized infant formula, comprising adding to a pasteurized infant formula an amount of nonhuman mammalian milk osteopontin, which is purified and obtained from bovine milk, sufficient to provide 50-250 mg/liter of the osteopontin, wherein the improved pasteurized infant formula is a bovine milk based infant formula.


SEQUENCE LISTING (2 sequences)

SEQ ID NO: 1 - protein, 298 aa, Homo sapiens
IPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 2 - protein, 262 aa, Bos taurus
LPVKPTSSGSSEEKQLNNKYPDAVAIWLKPDPSQKQTFLAPQNSVSSEETDDNKQNTLPSKSNESPEQTDDLDDDDDNSQDVNSNDSDDAETTDDPDHSDESHHSDESDEVDFPTDIPTIAVFTPFIPTESANDGRGDSVAYGLKSRSKKFRRSNVQSPDATEEDFTSHIESEEMHDAPKKTSQLTDHSKETNSSELSKELTPKRKDKNKHSNLIESQENSKLSQEFHSLEDKLDLDHKSEEDKHLKIRISHELDSASSEVN